(19) United States
(12) Patent Application Publication
(10) Pub. No.: US 2026/0181045 A1
(43) Pub. Date: Jun. 25, 2026

(54) Specialized 5G Core Network Functions for Medical Traffic

(71) Applicant: AT&T Intellectual Property I, L.P., Atlanta, GA (US)

(72) Inventors: Joseph Soryal, Glendale, NY (US); Venson Shaw, Kirkland, WA (US); Jason Chang, Decatur, GA (US)

(73) Assignee: AT&T Intellectual Property I, L.P., Atlanta, GA (US)

(21) Appl. No.: 18/986,895

(22) Filed: Dec. 19, 2024

(51) Int. Cl. H04L 67/12; G16H 10/00

(52) U.S. Cl. CPC H04L 67/12; G16H 10/00

(57) ABSTRACT

Concepts and technologies disclosed herein are directed to generating medical network functions that provide functionality tailored for handling medical devices and medical-related traffic from user devices that is beyond that provided by network functions of a next generation (“5G”) core network. Functionalities of a plurality of network functions of a 5G core network can be determined. The functionalities of the plurality of network functions can be duplicated to generate replicated network functions. Each of the replicated network functions can be integrated with a corresponding medical function to generate a plurality of medical network functions. The plurality of medical network functions can be deployed to the 5G core network.

### BACKGROUND

The development and deployment of next generation (“5G”) networks have revolutionized wireless communication, offering significantly faster data rates, lower latency, and improved reliability compared to previous generations of mobile networks. A critical feature of 5G technology is its unified core network, which provides a common platform to support a diverse range of devices and applications. While this unified architecture allows for simplified management and broad scalability, it may present challenges when addressing the specific needs of devices and applications that demand ultra-reliable, low-latency communication (“URLLC”) and heightened security, such as medical devices and communications involving medical information. Medical devices including wearable, implantable, and ingestible technologies generate and transmit sensitive medical data that require high-speed, secure, and reliable communication with minimal latency to ensure accuracy, uninterrupted monitoring, and immediate action in critical scenarios. However, the current 5G network infrastructure processes and routes traffic from medical devices using the same core elements as other 5G network traffic without distinguishing between the varying needs of connected devices and applications.

### SUMMARY

Concepts and technologies disclosed herein are directed to providing a medical functions core manager that generates medical network functions that provide functionality, beyond that provided by network functions of a 5G core network, tailored for handling medical devices and medical-related traffic from user devices. According to one aspect disclosed herein, a system can include a processor and a memory. The memory can store instructions for a medical functions core manager that, when executed by the processor, cause the processor to perform operations. In particular, the system can determine functionalities of a plurality of network functions of a 5G core network. The system can duplicate the functionalities of the plurality of network functions to generate replicated network functions. Each of the replicated network functions can be integrated with a corresponding medical function to generate a plurality of medical network functions. The system can deploy the plurality of medical network functions to the 5G core network. The plurality of medical network functions can provide functionality that extends beyond the functionalities of the plurality of network functions.

The system can also launch a plurality of virtual machines onto the 5G core network such that one of the plurality of virtual machines is launched in association with each of the plurality of network functions. The system can deploy one of the plurality of medical network functions to each of the plurality of virtual machines launched in association with a corresponding one of the plurality of network functions.

Data from a user device determined to be a medical device can be routed through at least a portion of the plurality of medical network functions instead of the plurality of network functions. Data determined to be associated with medical information can also be routed through at least a portion of the plurality of medical network functions instead of the plurality of network functions.

It should be appreciated that the above-described subject matter may be implemented as a computer-controlled apparatus, a computer process, a computing system, or as an article of manufacture such as a computer-readable storage medium. These and various other features will be apparent from a reading of the following Detailed Description and a review of the associated drawings.

Other systems, methods, and/or computer program products according to embodiments will be or become apparent to one with skill in the art upon review of the following drawings and detailed description. It is intended that all such additional systems, methods, and/or computer program products be included within this description, be within the scope of this disclosure.

### BRIEF DESCRIPTION OF THE DRAWINGS

FIG. 1 is a block diagram illustrating aspects of an illustrative operating environment for various concepts and technologies disclosed herein.

FIG. 2 is a flow diagram illustrating aspects of a method for generating a medical network function, according to an illustrative embodiment of the concepts and technologies disclosed herein, according to an illustrative embodiment.

FIG. 3 is a flow diagram illustrating aspects of a method for processing a communication from a user device via medical network functions of a 5G core network, according to an illustrative embodiment.

FIG. 4 is a block diagram of an example network, according to an illustrative embodiment

FIG. 5 is a block diagram illustrating a computer system configured to provide the functionality in accordance with various embodiments of the concepts and technologies disclosed herein.

FIG. 6 is a block diagram of a mobile device and components thereof, according to an illustrative embodiment.

FIG. 7 is a block diagram illustrating a cloud computing platform capable of implementing aspects of the concepts and technologies disclosed herein.

FIG. 8 is a diagram illustrating a machine learning system capable of implementing aspects of the embodiments disclosed herein, according to an illustrative embodiment.

### DETAILED DESCRIPTION

The concepts and technologies disclosed herein provide a medical functions core manager that generates medical network functions that provide functionality, beyond that provided by network functions of a 5G core network, tailored for handling medical devices and/or medical-related traffic from user devices. To generate the medical network functions, the medical functions core manager can replicate the functionality of the network functions of the 5G core network and integrate the replicated network functions with medical functions that include logic to enhance and extend the capability of the 5G core network to process, route, and manage medical-related traffic and medical devices. The medical functions core manager can create and provision virtual machines or containers with sufficient resources to run the medical network functions. The medical functions core manager can launch the virtual machines and/or containers in the 5G core network and deploy the medical network functions onto the virtual machines and/or containers. This setup allows flexibility in the deployment of medical functions, which can be scaled or selectively implemented based on network requirements.

To determine if a communication from a user device should be routed through the medical network functions, a gNodeB (“gNB”) of a radio access network (“RAN”) can determine whether the communication is associated with a medical flag. If a medical flag is present, the classifier can route the communication through the medical network functions to provide enhanced capabilities, such as low-latency processing and compliance with medical data regulations, to the communication. If no medical flag is detected, the classifier can analyze the content of the communication to determine if the content is associated with medical information. When medical content is detected, the communication is routed through the medical network functions, which are optimized to handle such traffic securely and efficiently.

While the subject matter described herein is presented in the general context of program modules that execute in conjunction with the execution of an operating system and application programs on a computer system, those skilled in the art will recognize that other implementations may be performed in combination with other types of program modules. Generally, program modules include routines, programs, components, data structures, and other types of structures that perform particular tasks or implement particular abstract data types. Moreover, those skilled in the art will appreciate that the subject matter described herein may be practiced with other computer system configurations, including hand-held devices, multiprocessor systems, microprocessor-based or programmable consumer electronics, minicomputers, mainframe computers, and the like.

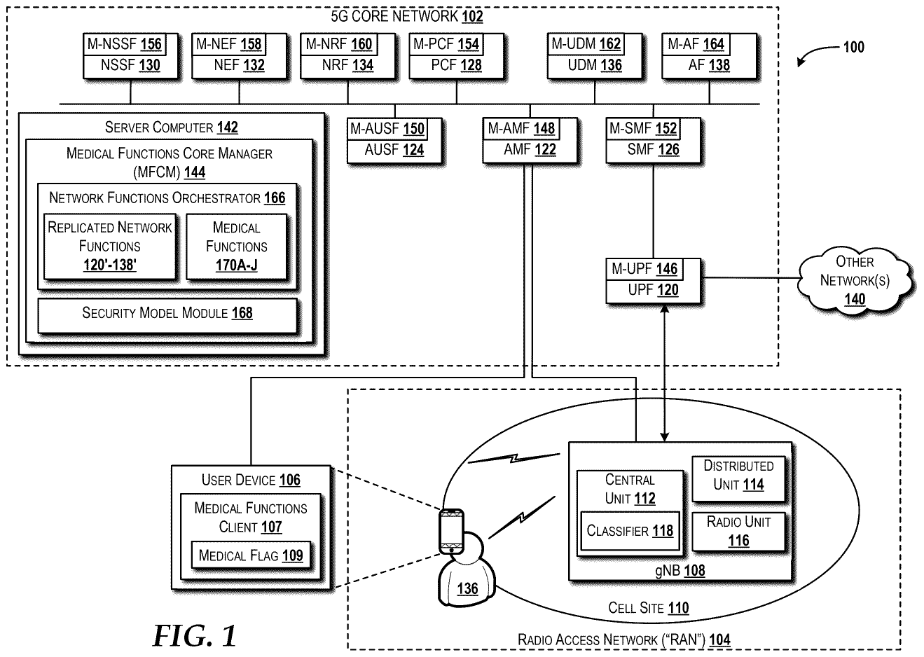

While connections are shown between some of the components illustrated in FIG. 1, it should be understood that some, none, or all of the components illustrated in FIG. 1 can be configured to interact with one another to carry out various functions described herein. Thus, it should be understood that FIG. 1 and the following description are intended to provide a general understanding of a suitable environment in which various aspects of embodiments can be implemented and should not be construed as being limiting in any way.

Turning now to FIG. 1, aspects of an operating environment 100 for various embodiments of the concepts and technologies disclosed herein will be described, according to an illustrative embodiment. The operating environment 100 includes a 5G core network 102, a 104, and a user device 106. The RAN 104 serves as an intermediary that connects the user device 106 to the 5G core network 102. The RAN 104 can be configured in accordance with Third Generation Partnership Project (“3GPP”) technical specifications for next generation (“5G”) RAN architectures. As such, in some embodiments, the RAN 104 can include a radio access node referred to as a gNB 108. Although only one gNB is shown in the illustrated example of the RAN 104 for a simplified representation, it should be understood by those skilled in the art that a plurality of gNBs may be deployed as part of the RAN 104 to enable broader coverage and capacity. Those skilled in the art will appreciate the applicability of the concepts and technologies disclosed herein to other RAN architectures or variations of the aforementioned RAN architectures.

The gNB 108 can provide a radio/air interface over which user equipment (“UE”), such as the user device 106, can connect to the RAN 104. The gNB 108 provides a coverage area, known as a cell site 110, within which the gNB 108 manages connectivity of devices. A mobile network operator (“MNO”) (not shown) can install the gNB 108 to provide network access for the user device 106 and/or other devices (not shown) in specific geographic locations. The gNB 108 operates by establishing a wireless connection that facilitates data transmission and reception between the user device 106 and the 5G core network 102. According to embodiments, the gNB 108 includes a central unit 112, a distributed unit 114, and a radio unit 116. The central unit 112 serves as the centralized control point that manages resource allocation, traffic management, and connection establishment. The central unit 112 can manage the connection between the user device 106 and the 5G core network 102 as well as the connection between the gNB 108 and the 5G core network 102. The central unit 112 can also facilitate handovers and coordination between different gNBs in the RAN 104. According to embodiments, the central unit 112 includes a classifier 118 that detects whether incoming traffic from a device, such as the user device 106, is associated with an indicator, such as a medical flag 109, specifying that the user device 106 is a medical device. If a medical flag 109 is not detected, the classifier 118 can determine whether content of the incoming traffic from a device, such as the user device 106, is associated with medical information. If the classifier 118 detects a medical flag 109 or content associated with medical information, the classifier 118 can determine that the incoming traffic should be routed through one or more of medical network functions 146-162 of the 5G core network 102, as discussed further below. The distributed unit 114 of the gNB 108 acts as an intermediate layer handling baseband signal processing, protocol termination, data forwarding, and synchronization. The radio unit 116 is responsible for wireless signal transmission and reception. In particular, the radio unit 116 converts baseband signals to radio frequency (“RF”) signals ensuring efficient coverage, signal quality, and capacity.

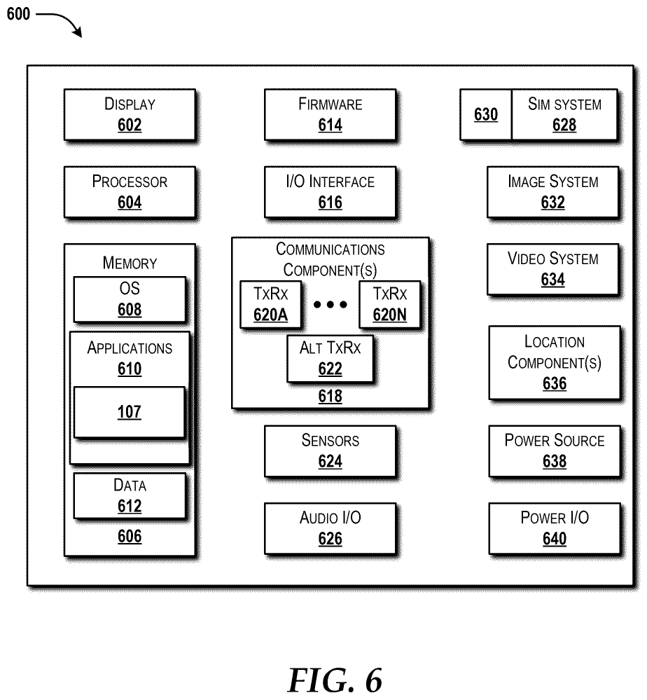

The user device 106 can be a wearable device, an implanted device (e.g., Internet of Bodies (“IoB”) device), a cellular phone (e.g., a feature phone or smartphone), a mobile computing device, a tablet computing device, a portable television, a portable video game console, or any other computing device that includes one or more radio access components that are capable of connecting to and communicating with one or more RANs, such as the RAN 104, via one or more radio access components. In some embodiments, the user device 106 can include an integrated or external radio access component that facilitates wireless communication with one or more RANs, such as the RAN 104. The user device 106 can wirelessly communicate with one or more RANs over a radio/air interface in accordance with one or more radio access technologies (“RATs”). The user device 106 may also initiate, receive, and maintain voice calls with one or more other voice-enabled telecommunications devices, such as other mobile devices or landline devices (not shown). The user device 106 may also exchange Short Message Service (“SMS”) messages, Multimedia Message Service (“MMS”) messages, email, and/or other messages with other devices (not shown). Additional details regarding the communication components of the user device 106 will be described below with reference to FIG. 6.

According to various embodiments of the concepts and technologies disclosed herein, the user device 106 is a medical device such as, for example, a wearable health monitor such as a smartwatch, fitness tracker, smart glasses, smart clothing, or other type of specialized wearable monitor that measures vital signs such as heart rate, blood pressure, glucose levels, and oxygen saturation; a medical implantable device such as a pacemaker, neurostimulator, cochlear implant, insulin pumps, orthopedic implants, electronic tattoos that monitor body metrics, subdermal microchips, or any other device implantable into the body to monitor health, improve bodily functions, and/or manage medical conditions; ingestible medical device such as smart pills, capsule endoscopy, and or ingestible biosensors; remote patient monitoring devices such as pulse oximeters, electrocardiogram monitor, blood pressure cuffs, and the like; mobile medical equipment such as equipment found in ambulances, portable ultrasound devices, defibrillators, and the like; surgical robots and tele-surgery equipment; smart diagnostic equipment such as MRIs, CT scanners, and x-ray machines; and/or any other type of medical device that is capable of communicating via the RAN 104. According to embodiments, medical versions of the user device 106 can execute a medical functions client 107 that associates a medical flag 109 with data (e.g., voice calls, video, messages, or any other type of communication) from the user device 106 to be transmitted via a RAN, such as the RAN 104, to a 5G core network 102. The medical functions client 107 could alternatively or additionally associate a medical flag 109 with a request sent by the user device 106, such as a radio resource control (“RRC”) connection request, an attach request, or a registration request, to establish a signaling connection with the gNB 108. According to embodiments, the medical flag 109 indicates that the request or data from the user device 106 is from a medical device and/or is related to medical information. For example, the medical flag 109 can be, but is not limited to, an identifier or tag inserted within an application-level payload of a communication indicating medical functionality of the user device 106 or an indicator included within a field of one of a radio resource control connection request, an attach request, or a registration request specifying the device type of the user device 106 as medical. As discussed further herein, the classifier 118 of the gNB 108 can detect the medical flag 109 associated with the request and/or traffic received from the user device 106 and, in response, can determine to route the traffic through one or more of the medical network functions 146-162 of the 5G core network 102 to optimize routing, bandwidth allocation, and security of the traffic.

The user device 106 forms a radio access network connection with the gNB 108, which is connected to a User Plane Function (“UPF”) 120 of the 5G core network 102 over a network interface such as, for example, an N3 interface. The UPF 120 connects to one or more other networks 140 over a network interface such as, for example, an N6 interface. The other networks 140 may be a data network used to provide an operator service or may be outside the scope of the standardization of the 3GPP technical specifications, such as the Internet, a network used to provide third party service, and/or an edge computing network or resource such as a Mobile Edge Computing (“MEC”) network. The user device 106 also connects to an Access and Mobility Management Function (“AMF”) 122 of the 5G core network 102. The AMF 122 is responsible for registration of UEs, such as the user device 106, upon connection to the 5G core network 102, authentication and authorization of access requests, mobility management of the UEs, and coordinating communication between the UEs and the rest of the 5G core network 102. For example, the AMF 122 can communicate with an Authentication Server Function (“AUSF”) 124 to authenticate that a particular UE, such as the user device 106, has authorization to access the 5G core network 102 and can communicate with a Session Management Function (“SMF”) 126 to manage data sessions and set up and modify user sessions for the user device 106, as discussed further below. The AMF 122 can also work with a Policy Control Function (“PCF”) 128 to enforce policy rules on the 5G core network 102 and with a Network Slice Selection Function (“NSSF”) 130 to determine the appropriate network slice for a particular UE or service, as also discussed further below.

In addition to the UPF 120, the AMF 122, the AUSF 124, the SMF 126, the PCF 128, and the NSSF 130, the 5G core network 102 can include other network functions such as at least one Network Exposure Function (“NEF”) 132, Network Repository Function (“NRF”) 134, Unified Data Management Function (“UDM”) 136, and Application Function (“AF”) 138 (collectively referred to herein as “network functions 120-138”). While 3GPP has defined some of these network functions, these network functions may be split into greater granularity to perform specific functions, may be combined, and/or additional functions may be added by the time the MNO deploys a live 5G core network. As such, the 5G core network 102 is intended to encompass any and all 5G core network functions that are currently defined in technical specifications currently available and revisions thereof made in the future.

Each of the network functions of the 5G core network 102 can be implemented either as a network element on a dedicated hardware, as a software instance running on a dedicated hardware, or as a virtualized function instantiated on an appropriate platform, such as a cloud infrastructure, as is appreciated by those skilled in the art. When one of these network functions initiates, the network function can register with the NRF 134 and provide information such as supported services of the network function, interface endpoints, capacity, load status, and other operational metrics. Network functions can query the NRF 134 to discover other network functions of the 5G core network 102.

The AUSF 124 is responsible for authenticating UEs attempting to connect to the 5G core network 102. The AUSF 124 can receive authentication requests initiated by the AMF 122 when a UE, such as the user device 106, connects to the 5G core network 102 and can access UE credentials stored in the UDM 136 for authentication purposes. According to embodiments, the UDM 136 manages subscriber data, such as user profiles, subscription information, and authentication credentials. The UDM 136 can retrieve credentials and subscription data associated with the UE 106 from a Home Subscriber Server (“HSS”) and can pass the information to the AUSF 124.

The SMF 126 is a network function that is responsible for setting up, modifying, and tearing down data paths between the RAN 104 and the UPF 120. The SMF 126 handles the allocation and management of IP addresses that are assigned to a UE, such as the user device 106, as well as the selection of a UPF, such as the UPF 120, for traffic associated with a particular session of the user device 106. The SMF 126 can also communicate with other functions, in a service based view, through a service based interface denoted as Nsmf.

After a UE, such as the user device 106, is authenticated, the NSSF 130 can be contacted by the AMF 122 to determine the appropriate network slice for the user device 106. The NSSF 130 is responsible for facilitating the selection and allocation of network slices to meet the varying requirements of different services, applications, and users. The NSSF 130 also can keep track of which slices are available in the network and the current capacity of the slices. The NSSF 130 processes requests from the AMF 122 to determine the most suitable network slice for an incoming connection based on service requirements, operator policies, and network conditions.

Once the network slice for the user device 106 is selected, the AMF 122 can request that the SMF 126 set up a data session for the user device 106. The SMF 126 can instruct the UPF 120 to establish a user data path for the user device 106. According to embodiments, the PCF 128 supplies policy rules to the SMF 126 for handling network operations, user traffic, quality of service (“QoS”), and charging. The SMF 126 can use the information from the PCF 128 to apply appropriate policies on the data session for the user device 106 and can forward configurations corresponding to the policies to the UPF 120 for use when routing and forwarding user data packets to external data networks, such as the other networks 140. After the data session for the user device 106 is set up and policies are applied, the UPF 120 forwards the data traffic from the user device 106 to the intended destination through the appropriate network slice.

The NEF 132 of the 5G core network 102 allows servers, functions, and other entities such as those outside a trusted domain to have exposure to services and capabilities within the 5G core network 102. In one such example, the NEF 132 can act much like a proxy between an application server outside the 5G core network 102 and network functions within the 5G core network 102, such as the AMF 122, the SMF 126, and the PCF 128, so that an external application server can provide information that may be of use in the setup of the parameters associated with a data session. The NEF 132 can communicate with other network functions through a service based Nnef network interface. The NEF 132 may also have an interface to non-3GPP functions.

An AF, such as the AF 138, serves as an intermediary between external applications or services and the 5G core network 102. The AF 138 allows external applications to interact with the 5G core network 102 by bridging the requirements of third-party services and the capabilities of the 5G core network 102 while ensuring security and compliance. The AF 138 may interact with network functions via the NEF 132 to request or configure certain policies. The AF 138 can also interact with functions such as the PCF 128 to provide application specific input into policy and policy enforcement decisions. Additionally, or alternatively, the NEF 132 can interact with the SMF 126, PCF 128, and other network functions to enforce requests made by the AF 138. For example, if a third-party service requires low latency for real-time video, the AF 138 can request the same, and the NEF 132 can communicate this requirement to the PCF 128, which adjusts the QoS policies accordingly.

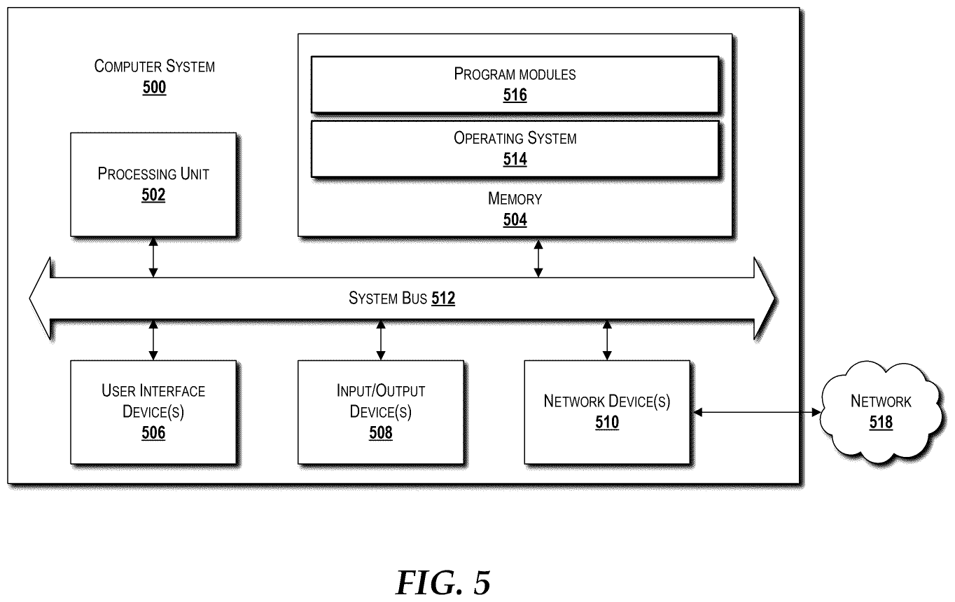

According to embodiments, the 5G core network 102 also includes a server computer 142 hosting a medical functions core manager (“MFCM”) 144. The MFCM 144 can be a software module executed by processing unit (herein also referred to as a “processor”) 502 of the server computer 142, illustrated and discussed further with regards to FIG. 5, or can be hardware modules or combinations of hardware and software that perform the operations described herein. The MFCM 144 can create and deploy a plurality of virtual machines and/or containers in association with the network functions 120-138 of the 5G core network 102. According to embodiments, a virtual machine and/or container can be deployed to each of the network functions 120-138 of the 5G core network 102. The virtual machines and/or containers deployed by the MFCM 144 can run a medical-UPF (“M-UPF”) 146, a medical-AMF (“M-AMF”) 148, a medical-AUSF (“M-AUSF”) 150, a medical-SMF (“M-SMF”) 152, a medical-PCF (“M-PCF”) 154, a medical-NSSF (“M-NSSF”) 156, a medical-NEF (“M-NEF”) 158, a medical-NRF (“M-NRF”) 160, a medical-UDM (“M-UDM”) 162, and a medical-AF (“M-AF”) 164 (hereinafter collectively referred to as “medical network functions 146-164”) generated by the MFCM 144. As discussed further herein, each of the medical network functions 146-164 can be deployed one of the virtual machines/containers as a virtualized network function and/or a containerized microservice that replicates the functionality of a corresponding one of the network functions 120-138 and provides additional functionality that is tailored for handling medical devices and/or medical-related traffic associated with the 5G core network 102.

According to various embodiments of the concepts and technologies disclosed herein, the functionality of the server computer 142 may be provided by one or more server computers, application servers, web servers, data processing resources, gateway devices, routers, other computing systems, and the like. It should be understood that the functionality of the server computer 142 may be provided by a single device, by two or more similar devices, and/or by two or more dissimilar devices. For purposes of describing the concepts and technologies disclosed herein, the server computer 142 is described herein as an application server. It should be understood that this embodiment is illustrative, and should not be construed as being limiting in any way.

The MFCM 144 can be a software module executed by a processor of the server computer 142 or can be hardware modules or combinations of hardware and software that perform the operations described herein. Details regarding the components of the server computer 142 will be described below with reference to FIG. 5. The MFCM 144 can include a network functions orchestrator 166 and a security model module 168. According to embodiments, the network functions orchestrator 166 can determine the functionality of each of the network functions 120-138 in preparation for creating the medical network functions 146-164 that replicate at least a portion of the functionality of the network functions 120-138 hosting the medical network functions 146-164. To determine these functionalities, the network functions orchestrator 166 can inspect the configuration of the network functions 120-138 by examining the configuration files and/or management interfaces associated with the network functions 120-138. The network function orchestrator 166 can additionally or alternatively interrogate the network functions 120-138 using application programming interfaces (“APIs”) exposed by the network functions 120-138. Moreover, the network functions orchestrator 166 can observe interactions between the network functions 120-138, conduct testing and simulations on the network functions 120-138, and/or examine logs and metrics associated with the network functions 120-138 to determine the functionality of each.

Regardless of how the functionality of each of the network functions 120-138 is determined, the network functions orchestrator 166 can use the information about the functionality to generate replicated network functions 120′-138′, each of which duplicates the functionality of a corresponding one of the network functions 120-138 of the 5G core network 102. For instance, considering the UPF 120 as the network function, the network functions orchestrator 166 can create a design document outlining the components and interfaces of the UPF 120 based on the information about the functionality of the UPF 120 attained using one or more of the processes discussed above. The network functions orchestrator 166 can use at least the information about the functionality of the UPF 120 and the design document to develop the replicated network function 120′ as an application or a set of microservices that replicates the functionality of the UPF 120. A similar process can be used by the network functions orchestrator 166 to develop the replicated network functions 122′-138′ for the remaining network functions 122-138.

In addition to the replicated network functions 120′-138′, the network functions orchestrator 166 can also generate a medical function, such as one of medical functions 170A-J, associated with each of the replicated network functions 120′-138′. Each of the medical functions 170A-J can include logic to enhance the capability of the 5G core network 102 to process, route, and manage medical-related traffic and medical devices. For instance, the medical functions 170A-J can include mechanisms to provide traffic prioritization tailored for medical devices and medical-related traffic, low-latency processing algorithms for ultra-reliable low-latency communication (“URLLC”) for medical-related traffic, and enhanced security protocols to ensure compliance with medical data regulations (e.g., HIPAA, GDPR, etc.) for medical devices and medical-related traffic. According to embodiments, the security model module 168 of the MFCM 144 can utilize a generative artificial intelligence (“AI”) model to monitor, analyze, and summarize changes in data protection regulations regarding health and medical information (e.g., HIPAA, GDPR, local regulations, etc.) to ensure that the medical functions 170A-J and the medical network functions 146-164 comply with current regulations. For example, the security model module 168 can use web scraping, API integration, and the like to monitor official regulatory websites, legislative announcements, and compliance forums for updates related to laws associated with protection regulations regarding health and medical information. When updates/modifications to regulations are detected, the security model module 168 can use the information gleaned from such sources to draft updated policies and procedures based on the new/modified regulations. The security model module 168 can then apply the changes to the medical functions 170A-J and/or communicate with the network functions orchestrator 166 to implement the changes to the configurations and/or policies of the medical network functions 146-164 of the 5G core network 102.

According to embodiments, the network functions orchestrator 166 can integrate each of the medical functions 170A-J with a corresponding one of the replicated network functions 120′-138′ to generate the medical network functions 146-164. As discussed further below, the medical network functions 146-164 can be deployed to the 5G core network 102 by the network functions orchestrator 166 to provide, for medical-related traffic and devices, functionality similar to that of the network functions 120-138 with enhancements designed specifically for handling the requirements of such traffic and devices.

The network functions orchestrator 166 can create and provision virtual machines with sufficient resources to run the medical network functions 146-164. According to embodiments, the network function orchestrator 166 can use tools like VMWARE, HYPER-V, or KVM to create the virtual machines. Alternatively or additionally, the network functions orchestrator 166 may provision containers with sufficient resources to run the medical network functions 146-164. The network functions orchestrator 166 can use tools like DOCKER or CONTAINERD to create the containers. Once the virtual machines and/or containers are prepared, the network functions orchestrator 166 can launch the virtual machines and/or containers onto the 5G core network 102 and deploy the medical network functions 146-164 onto the virtual machines and/or containers. In particular, the network functions orchestrator 166 can deploy the M-UPF 146 to the UPF 120, the M-AMF 148 to the AMF 122, the M-AUSF 150 to the AUSF 124, the M-SMF 152 to the SMF 126, the M-PCF 154 to the PCF 128, the M-NSSF 156 to the NSSF 130, the M-NEF 158 to the NEF 132, the M-NRF 160 to the NRF 134, the M-UDM 162 to the UDM 136, and the M-AF 164 to the AF 138. Although a medical network function is illustrated as being deployed to each of the network functions 120-138, it should be appreciated that a medical network function can be deployed to fewer than all of the network functions 120-138 based, for example, on the specific requirements of the 5G core network 102. Additionally, although only one each of the network functions 120-138 is illustrated in FIG. 1, it should be understood that multiples of one or more of the network functions 120-138 can be deployed in the 5G core network 102 based on, for example, network capacity and geographic coverage. According to embodiments, a corresponding one of the medical network functions 146-164 can be deployed to each multiple of a network function deployed in the 5G core network 102.

Each of the medical network functions 146-164 is designed to complement and extend the functionality of a corresponding one of the network functions 120-138 of the 5G core network 102 by incorporating features specifically designed to support requirements of medical-related traffic and devices. According to embodiments, the M-UPF 146 can implement low-latency data routing and prioritization for medical-related traffic. The M-UPF 146 can also provide specialized packet handling for healthcare protocols to ensure consistent data delivery for medical devices. For traffic directed to a medical device or medical-related traffic directed to any type of device, the M-UPF 146 can act as an extra firewall examining and filtering out any traffic determined to be potentially malicious. The M-UPF 146 can also act as a firewall regarding traffic associated with a medical device or medical-related traffic associated with any type of device destined for a network outside the 5G core network 102 by filtering and blocking any outgoing traffic determined to be inadvertently releasing sensitive information associated with a user of the medical device/device to one or more unauthorized entities.

Considering that the AMF 122 is the first network function contacted by a device, such as the user device 106, during initial registration with the 5G core network 102, the M-AMF 148 can interface with the M-UPF 146 and the M-SMF 152 to orchestrate the setup of routing medical-related traffic and traffic from medical devices via the medical network functions 146-164. The M-AMF 148 can also enhance signaling efficiency to minimize connection setup times for medical devices or devices determined to be associated with medical-related traffic. In addition, the M-AMF 148 can optimize mobility management for medical devices or devices determined to be associated with medical-related traffic to ensure seamless handovers between gNBs.

According to embodiments, the M-AMF 148 provides enhanced tracking capability of a medical device or device associated with medical-related traffic. Conventionally, in order to coordinate mobility management for a device, the AMF 122 tracks the location of the device at a broad level, such as within the boundaries of a tracking area where the device is registered. The M-AMF 148 can enhance this tracking capability by determining a more precise location of a device versus what is typically determined by the AMF 122. For example, the M-AMF 148 can request GPS location information from the device; determine proximity of the device to other devices for which location is known based on, for example, one or more signal strength indicators received by the device from the other devices; receive information from inertial sensors in the device to track movement and estimate the location of the device; and the like. The M-AMF 148 can leverage this precise location information to enhance the quality of service provided to the device as well as a user of the device. For instance, the M-AMF 148 can use the precise location information to optimize which gNB, such as the gNB 108, serves the device and predict next gNBs to serve the device as the device moves, which minimizes handover delays and provides optimal signal strength for the device. The M-AMF 148 can also forward the precise location information to emergency responders and/or notify nearby caregivers or medical professionals within the vicinity of the user if a determination is made that a user of the device is involved in a critical health event.

The M-AUSF 150 can provide extra authentication for devices associated with medical-related traffic attempting to connect to the 5G core network 102 that ensures secure access to the 5G core network 102 while maintaining compliance with the heightened healthcare data protection regulations. According to embodiments, the M-AUSF 150 supports advanced, hardware-specific authentication protocols tailored for medical devices and devices associated with medical-related traffic as well as multi-factor authentication protocols. Additionally, the M-AUSF 150 can incorporate lightweight authentication protocols, such as elliptic curve cryptography, for efficient yet secure authentication of medical devices or devices associated with medical-related traffic with limited processing power, such as wearable devices and IoB devices. According to embodiments, the M-AUSF 150 implements authentication processes that align with healthcare data protection laws by employing, for example, encrypted credentials for compliance with HIPAA, GDPR, and/or regional medical standards, logging authentication attempts for audit purposes and regulatory reviews, ensuring that consent is obtained from a user of the device before transmitting medical-related data with external systems, and the like.

The M-UDM 162 serves as a centralized data repository for subscription data, subscriber policy data, sessions, contexts, and application states and can enhance user and subscription data management for medical devices. In addition to generic device profiles and authentication data stored by the UDM 136, the M-UDM 162 stores healthcare-specific data such as device certifications, patient-linked profiles including health information associated with a user of the device (e.g., medical history, medications, diseases, etc.), medical contact information including doctor and hospital information associated with the user as well as information about which doctor to contact based on a particular medical situation, consent records, and/or any other medical-related information associated with a user of the device. The M-UDM 162 can ensure compliance with regulations by segregating medical data from other subscription records and encrypting the medical data to prevent unauthorized access.

The M-SMF 152 can establish and manage data sessions for medical devices and medical-related traffic. In addition to the session setup and resource allocation provided by the SMF 126, the M-SMF 152 integrates advanced algorithms to prioritize the setup and maintenance of sessions for medical devices and devices associated with medical-related traffic. The M-SMF 152 can interface with the M-AMF 148 and M-UPF 146 to ensure that Quality of Service (“QoS”) parameters, such as low latency and high reliability, are dynamically applied to medical-related traffic. According to embodiments, the M-SMF 152 also collaborates with the M-UDM 162 to retrieve profile and policy information associated with a medical device and/or user of the medical device. The M-SMF 152 can use the information retrieved from the M-UDM 162 when deciding how to route traffic associated with a medical device and medical-related traffic associated with any type of device. For example, for a call determined by the classifier 118 of the gNB 108, as discussed further below, to indicate an overdose of medicine, the M-SMF 152 can determine to establish a data session for the call with a doctor corresponding to doctor information retrieved from the M-UDM 162.

Additionally, the M-SMF 152 can provide enhanced session continuity for medical devices. For instance, during mobility events, the M-SMF 152 ensures seamless transfer of ongoing medical sessions to new gNBs or M-UPFs by preemptively reserving resources in anticipation of movement. This minimizes disruptions during procedures like remote surgeries or telemonitoring. The M-SMF 152 can also maintain session integrity by implementing specialized failover mechanisms, such as backup session pathways for critical traffic, ensuring that medical data transmission is not interrupted.

The M-PCF 154 can enhance the management of policies specific to medical traffic and devices. The M-PCF 154 dynamically generates policies tailored to the criticality and urgency of medical traffic. For example, the M-PCF 154 can integrate with hospital systems or electronic health record (“EHR”) platforms to categorize medical traffic based on its importance, such as emergency alerts versus routine monitoring data. Furthermore, the M-PCF 154 can enforce healthcare-specific policies, such as HIPAA-compliant data handling or prioritizing emergency traffic during network congestion. The M-PCF 154 can coordinate with the M-SMF 152 to ensure that policy decisions are reflected in resource allocation and with the M-AMF 148 to manage signaling flows according to medical priority. For example, during a network outage, the M-PCF 154 can override standard traffic priorities to preserve medical device connectivity, ensuring continuity of critical services.

The M-NSSF 156 can specialize in selecting network slices optimized for medical applications. Where the NSSF 130 may assign slices based on generic device profiles, the M-NSSF 156 evaluates the specific requirements of medical devices or traffic when assigning slices. The M-NSSF 156 can interface with the M-AMF 148 and M-PCF 154 to ensure that devices are placed on slices with predefined medical QoS guarantees, such as URLLC. In addition, the M-NSSF 156 can consider the traffic requirement of a device to determine what size slice to assign. For example, the M-NSSF 156 may assign a slice with adequate resources for smaller-scale communication, such as when providing for a medical device, and assign a larger slice for a hospital or healthcare provider, which often deals with large-scale data transmission, such as imaging, patient records, or remote surgeries.

The M-NSSF 156 can also dynamically adapt slice assignments based on real-time conditions. For instance, if a medical device transitions from routine monitoring to emergency status, the M-NSSF 156 can migrate the medical device to an emergency slice with higher priority and bandwidth. Additionally, the M-NSSF 156 can support multi-slice connectivity for complex healthcare workflows, enabling a single device to access separate slices for diagnostics, imaging, and communication concurrently.

The M-NEF 158 can enhance the exposure of network capabilities specifically for medical applications. Where the NEF 132 may provide generic APIs for third-party integration, the M-NEF 158 offers APIs tailored to healthcare use cases. These APIs can enable secure interaction between the 5G core network 102 and external medical platforms, such as hospital information systems, pharmaceutical information systems, doctor information systems, and/or Internet of Thing (“IoT”) platforms for wearable medical devices. The M-NEF 158 can include mechanisms to ensure compliance with healthcare regulations during API usage. For example, the M-NEF 158 can enforce granular access controls to prevent unauthorized access to sensitive medical data. Additionally, the M-NEF 158 can support real-time monitoring and notifications for critical events, such as abnormal vital signs detected by connected devices, and relay this information to authorized healthcare providers or emergency services.

The M-NRF 160 can serve as a registry for the medical network functions 146-158 and 162-164 of the 5G core network 102, ensuring discovery and interaction among components specialized for handling medical-related traffic and devices. The medical network functions 146-158 and 162-164 can register with the M-NRF 160 when the medical network functions 146-158 and 162-164 are instantiated on the 5G core network 102. The M-NRF 160 can include metadata tagging for registered functions, such as medical network functions 146-158 and 162-164, to indicate the functions suitability for medical traffic, such as compliance with URLLC requirements or HIPAA standards. The M-NRF 160 can work in conjunction with the M-NSSF 156 and M-PCF 154 to optimize network performance for medical devices. For example, the M-NRF 160 can provide the M-AMF 148 with real-time information about available medical slices and associated resources, enabling dynamic adjustments to device registrations. The M-NRF 160 can also support redundancy by identifying alternative medical functions during outages or congestion.

The M-AF 164 can provide application-layer support for healthcare workflows. The M-AF 164 can interface with external healthcare applications to ensure seamless integration with the 5G core network 102. For instance, the M-AF 164 can facilitate low-latency communication for a remote surgery application by requesting a dedicated network slice and ensuring that sufficient resources are allocated to maintain a reliable and secure connection.

The network functions orchestrator 166 can continuously monitor the performance of the medical network functions 146-164 to ensure service levels provided by the medical network functions 146-164 are met. If medical-related demands on the 5G core network 102 increase, the network functions orchestrator 166 can scale the medical network functions 146-164 vertically and/or horizontally to meet the demand. For example, the network functions orchestrator 166 can allocate more resources to an existing medical network function and/or initiate additional instances of one or more of the medical network functions 146-164 based on the demand experienced by the 5G core network 102. Similarly, if medical-related demands on the 5G core network 102 decrease, the network functions orchestrator 166 can reduce the amount of resources to an existing medical network function and/or delete instances of one or more of the medical network functions 146-164. Although more or less instances of the medical network functions 146-164 can be instantiated based on the demand experienced by the 5G core network 102, at least one instance of each of the medical network functions 146-164 is maintained in a continuous operational state ready to process data or requests. In addition, according to embodiments, routes between the medical network functions 146-164 remain in a persistent state such that the routes remain established and operational to reduce connectivity issues and delays in transmission when handling medical-related traffic as well as to reduce the overhead of dynamic route discovery and setup when dealing with such traffic.

In order to ascertain whether a communication from a device, such as the user device 106, should be routed through one or more of the medical network functions 146-164, a determination is made whether the communication is from a medical device (i.e., whether the user device 106 is a medical device) or whether the communication is associated with medical information. As discussed above, the gNB 108 includes a classifier 118 that detects whether a communication from the user device 106 or a request received to establish communication between the user device 106 and the gNB 108 is associated with an indicator, such as a medical flag 109, specifying that the user device 106 is a medical device. If the classifier 118 detects a medical flag 109, the classifier 118 can determine that incoming communications from the user device 106 should be routed through one or more of the medical network functions 146-162 of the 5G core network 102 such that the communications are processed and managed by the enhanced capabilities of the medical network functions 146-162 tailored for handling communications from medical devices instead of the network functions 120-138. According to embodiments, the classifier 118 can also signal to one or more of the medical network functions 146-164 that the incoming communication is to be routed via one or more of the medical network functions 146-162 of the 5G core network 102.

In addition to identifying the user device 106 as a medical device, the medical flag 109 can also indicate a level of priority to be afforded a communication from the user device 106. For example, the medical flag 109 can indicate a high priority for a communication associated with an emergency medical situation. The level of priority associated with a medical flag 109 can be determined and assigned by the medical functions client 107 of the user device 106. For example, the medical functions client 107 can assign a high priority to the medical flag 109 based on detecting an anomaly in data gathered by the user device 106 indicating that a user may be involved in a medical emergency. The medical functions client 107 can also assign a high priority to the medical flag 109 based on information received from the gNB 108. For example, when the user device 106 sends a connection request, such as an RRC connection request, to the gNB 108, the gNB 108 can register the user device 106 with the 5G core network 102 and, in response, receive information from the 5G core network 102 about the user device 106. This information can include, but is not limited to, information from the M-UDM 162 indicating that a user of the user device 106 has recently undergone a medical procedure. If the medical functions client 107 then detects selection of the numbers “9” and “1” on the user device 106, the medical functions client 107 can set the priority of the medical flag 109 to high and prompt the user device 106 to send a communication with the medical flag 109 to the gNB 108 even prior to the dialed number being completed. According to embodiments, when the classifier 118 receives a request or communication associated with a medical flag indicating a high priority, the gNB 108 can put the M-UPF 146 on notice of a potential medical emergency. In response, the M-UPF 146 can pre-establish a standby route to emergency services, such as 911 services, and/or preferred healthcare providers or emergency contacts associated with a user of the user device 106 determined, for example, based on information from the M-UDM 162.

If, on the other hand, a medical flag 109 is not detected in the request or communication from the user device 106, the classifier 118 can determine whether content of the incoming communication from the user device 106 is associated with medical information. According to embodiments, the classifier 118 can perform data analysis and/or speech recognition on the communication from the user device 106 to determine whether the content is associated with medical information. Prior to determining content of any communication from a device, such as the user device 106, the classifier 118 would need to request and receive authorization from a user of the user device 106 to perform such analysis. If the classifier 118 determines that the content of a communication is associated with medical information, the classifier 118 can determine that the communication should be routed through one or more of the medical network functions 146-162 of the 5G core network 102 such that the communication is processed and managed by the enhanced capabilities of the medical network functions 146-162 tailored for handling communications from medical devices instead of the network functions 120-130. The classifier 118 can also signal to one or more of the medical network functions 146-164 that the incoming communication is to be routed via one or more of the medical network functions 146-162 of the 5G core network 102.

FIG. 1 illustrates one user device 106, one RAN 104, one gNB 108, one server computer 142, one of each network function 120-138, and one of each medical network function 146-164. It should be understood, however, that various implementations of the operating environment 100 can include one or more than one user device 106, one or more than one RAN 104, one or more than one gNB 108, one or more than one server computer 142, one or more than one of each network function 120-138, and one or more than one of each medical network function 146-164. As such, the illustrated embodiment should be understood as being illustrative, and should not be construed as being limiting in any way.

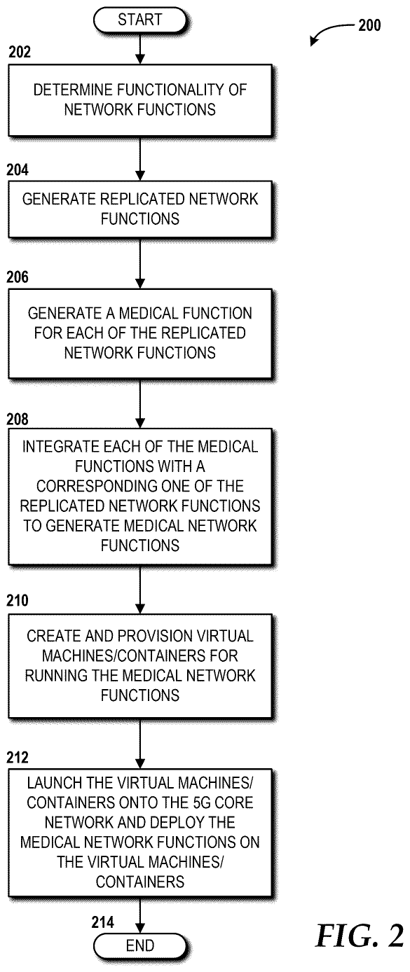

Turning now to FIG. 2, a flow diagram illustrating aspects of a method 200 for generating a medical network function, such as one of the medical network function 146-164, via the MFCM 144 hosted by the server computer 142 will be described, according to an illustrative embodiment of the concepts and technologies disclosed herein. It should be understood that the operations of the methods disclosed herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the concepts and technologies disclosed herein.

It also should be understood that the methods disclosed herein can be ended at any time and need not be performed in its entirety. Some or all operations of the methods, and/or substantially equivalent operations, can be performed by execution of computer-readable instructions included on a computer storage media, as defined herein. The term “computer-readable instructions,” and variants thereof, as used herein, is used expansively to include routines, applications, application modules, program modules, programs, components, data structures, algorithms, and the like. Computer-readable instructions can be implemented on various system configurations including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These states, operations, structural devices, acts, and modules may be implemented in software, in firmware, in special purpose digital logic, and any combination thereof. As used herein, the phrase “cause a processor to perform operations” and variants thereof is used to refer to causing a processor of a computing system or device, or a portion thereof, to perform one or more operations, and/or causing the processor to direct other components of the computing system or device to perform one or more of the operations.

For purposes of illustrating and describing the concepts of the present disclosure, operations of the methods disclosed herein are described as being performed alone or in combination via execution of one or more software modules, and/or other software/firmware components described herein. It should be understood that additional and/or alternative devices and/or network nodes can provide the functionality described herein via execution of one or more modules, applications, and/or other software. Thus, the illustrated embodiments are illustrative, and should not be viewed as being limiting in any way.

The method 200 will be described with reference to FIG. 1. The method 200 begins and proceeds to operation 202, where the server computer 142, via the network functions orchestrator 166 of the MFCM 144 hosted by the server computer 142, determines the functionality of each of the network functions 120-138 of the 5G core network 102. To determine these functionalities, the network functions orchestrator 166 can inspect the configuration of the network functions 120-138 by examining the configuration files and/or management interfaces associated with the network functions 120-138. The network functions orchestrator 166 can additionally or alternatively interrogate the network functions 120-138 using application programming interfaces (“APIs”) exposed by the network functions 120-138. Moreover, the network functions orchestrator 166 can observe interactions between the network functions 120-138, conduct testing and simulations on the network functions 120-138, and/or examine logs and metrics associated with the network functions 120-138 to determine the functionality of each.

From operation 202, the method 200 proceeds to operation 204, where the network functions orchestrator 166 can use the information about the functionality to generate replicated network functions 120′-138′, each of which duplicates the functionality of a corresponding one of the network functions 120-138 of the 5G core network 102. For instance, considering the UPF 120 as the network function, the network functions orchestrator 166 can create a design document outlining the components and interfaces of the UPF 120 based on the information about the functionality of the UPF 120 attained using one or more of the processes discussed above. The network functions orchestrator 166 can use at least the information about the functionality of the UPF 120 and the design document to develop the replicated network function 120′ as an application or a set of microservices that replicates the functionality of the UPF 120. A similar process can be used by the network functions orchestrator 166 to develop the replicated network functions 122′-138′ for the remaining network functions 122-138.

From operation 204, the method 200 proceeds to operation 206, where the network functions orchestrator 166 can generate a medical function, such as one of medical functions 170A-J, associated with each of the replicated network functions 120′-138′. Each of the medical functions 170A-J can include logic to enhance the capability of the 5G core network 102 to process, route, and manage medical-related traffic and medical devices. For instance, the medical functions 170A-J can include mechanisms to provide traffic prioritization tailored for medical devices and medical-related traffic, low-latency processing algorithms for ultra-reliable low-latency communication (“URLLC”) for medical-related traffic, and enhanced security protocols to ensure compliance with medical data regulations (e.g., HIPAA, GDPR, etc.) for medical devices and medical-related traffic.

From operation 206, the method 200 proceeds to operation 208, where the network functions orchestrator 166 can integrate each of the medical functions 170A-J with a corresponding one of the replicated network functions 120′-138′ to generate the medical network functions 146-164. According to embodiments, the medical network functions 146-164 include features that extend the capability of the 5G core network 102 to process, route, and manage medical-related traffic and medical devices. From operation 208, the method 200 proceeds to operation 210, where the network functions orchestrator 166 can create and provision virtual machines with sufficient resources to run the medical network functions 146-164. According to embodiments, the network functions orchestrator 166 can use tools like VMWARE, HYPER-V, or KVM to create the virtual machines. Alternatively or additionally, the network functions orchestrator 166 may provision containers with sufficient resources to run the medical network functions 146-164. The network functions orchestrator 166 can use tools like DOCKER or CONTAINERD to create the containers.

From operation 210, the method 200 proceeds to operation 212, where the network functions orchestrator 166 launches the virtual machines and/or containers onto the 5G core network 102 and deploy the medical network functions 146-164 onto the virtual machines and/or containers. In particular, the network functions orchestrator 166 can deploy the M-UPF 146 to the UPF 120, the M-AMF 148 to the AMF 122, the M-AUSF 150 to the AUSF 124, the M-SMF 152 to the SMF 126, the M-PCF 154 to the PCF 128, the M-NSSF 156 to the NSSF 130, the M-NEF 158 to the NEF 132, the M-NRF 160 to the NRF 134, the M-UDM 162 to the UDM 136, and the M-AF 164 to the AF 138. From operation 212, the method 200 proceeds to operation 214, where the operation 200 ends.

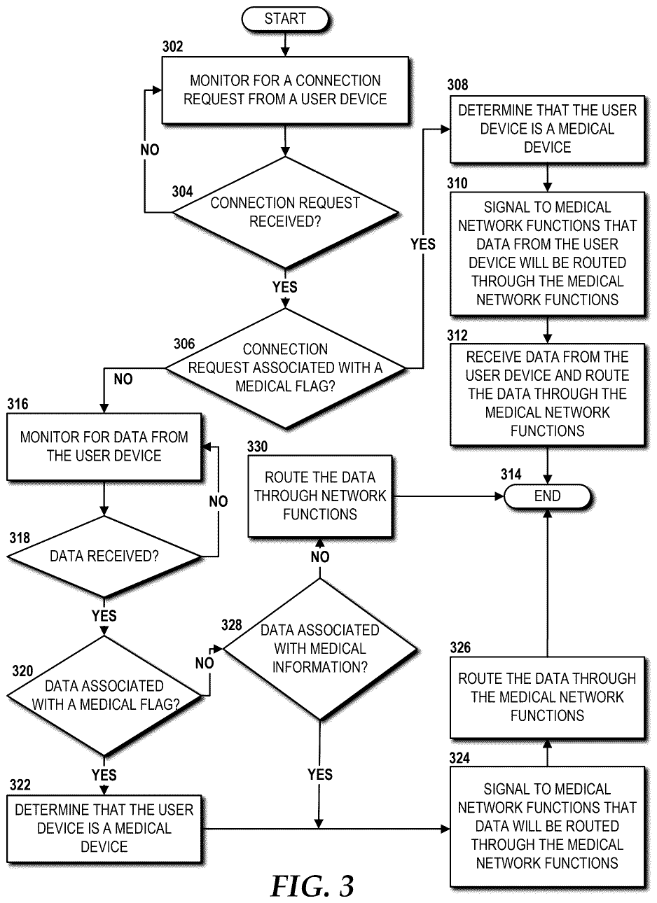

Turning now to FIG. 3, flow diagrams illustrating aspects of a method 300 for processing a communication from a user device, such as the user device 106, via the medical network functions 146-164 of the 5G core network 102 will be described, according to an illustrative embodiment of the concepts and technologies disclosed herein. It should be understood that the operations of the methods disclosed herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the concepts and technologies disclosed herein.

The method 300 will be described with reference to FIG. 1. The method 300 begins and proceeds to operation 302, where the gNB 108 monitors for a connection request from a user device, such as the user device 106. From operation 302, the method 300 proceeds to operation 304, where the gNB 108 determines whether a connection request is received from the user device 106. For example, the gNB 108 can determine whether an RRC connection request is received from the user device 106 to establish a signaling connection with the gNB 108. If a determination is made that a connection request from the user device 106 is not received, the method 300 proceeds back to operation 302, where the gNB 108 continues to monitor for a connection request from the user device 106. If, on the other hand, the gNB 108 determines that a connection request from the user device 106 has been received, the method 300 proceeds from operation 304 to operation 306. At operation 306, the gNB 108 determines, via the classifier 118, whether the connection request from the user device 106 is associated with a medial flag 109. For instance, the classifier 118 can determine whether a field of the connection request includes an identifier or tag indicating that the user device 106 is a medical device.

If, at operation 306, a determination is made that the connection request from the user device 106 is associated with a medical flag 109, the method 300 proceeds to operation 308, where the classifier 118 determines that the user device 106 is a medical device. From operation 308, the method 300 proceeds to operation 310, where the classifier 118 signals to one or more of the medical network functions 146-162 of the 5G core network 102 that data (e.g., voice calls, video, messages, or any other type of communication) originating from the user device 106 will be routed through one or more of the medical network functions 146-162 of the 5G core network 102 when received. From operation 310, the method 300 proceeds to operation 312, where the gNB 108 receives data from the user device 106 and routes the data through one or more of the medical network functions 146-162 of the 5G core network 102 such that the data is processed and managed by the enhanced capabilities of the medical network functions 146-162 tailored for handling communications from medical devices instead of the network functions 120-138. From operation 312, the method 300 proceeds to operation 314 where the method 300 ends.

Returning to operation 306, if a determination is made that the connection request from the user device 106 is not associated with a medical flag 109, the method 300 proceeds to operation 316, where the gNB 108 monitors for data (e.g., voice calls, video, messages, or any other type of communication) from the user device 106. From operation 316, the method 300 proceeds to operation 318, where the gNB 108 determines whether data from the user device 106 has been received. If a determination is made that data from the user device 106 is not received, the method 300 proceeds back to operation 316, where the gNB 108 continues to monitor for data from the user device 106. If, on the other hand, the gNB 108 determines that data from the user device 106 has been received, the method 300 proceeds from operation 318 to operation 320. At operation 320, the gNB 108 determines, via the classifier 118, whether the data from the user device 106 is associated with a medial flag 109. For instance, the classifier 118 can determine whether an identifier or tag is inserted within an application-level payload of the data indicating that the user device 106 is a medical device.

If, at operation 320, a determination is made that the data from the user device 106 is associated with a medical flag 109, the method 300 proceeds to operation 322, where the classifier 118 determines that the user device 106 is a medical device. From operation 322, the method 300 proceeds to operation 324, where the classifier 118 signals to one or more of the medical network functions 146-162 of the 5G core network 102 that the data will be routed through one or more of the medical network functions 146-162 of the 5G core network 102. From operation 324, the method 300 proceeds to operation 326, where the gNB 108 routes the data through one or more of the medical network functions 146-162 of the 5G core network 102 such that the data is processed and managed by the enhanced capabilities of the medical network functions 146-162 tailored for handling communications from medical devices instead of the network functions 120-138. From operation 326, the method 300 proceeds to operation 314 where the method 300 ends.

Returning to operation 320, if a determination is made that the data from the user device 106 is not associated with a medical flag 109, the method 300 proceeds to operation 328, where the classifier 118 determines whether content of the data is associated with medical information. According to embodiments, the classifier 118 can perform data analysis and/or speech recognition on the data from the user device 106 to determine whether the content of the data is associated with medical information. Prior to determining the content of any data from a device, such as the user device 106, the classifier 118 would need to request and receive authorization from a user of the user device 106 to perform such analysis. If the classifier 118 determines that the content of the data is associated with medical information, the method 300 can proceed from operation 328 to operations 324-326, as described above. If, on the other hand, the classifier 118 determines that the content of the data is not associated with medical information, the method 300 proceeds from operation 328 to operation 330, where the gNB 108 routes the data through the network functions 120-138 of the 5G core network 102 such that the data is processed as usual by the capabilities of the network functions 120-138. From operation 330, the method 300 proceeds to operation 314 where the method 300 ends.

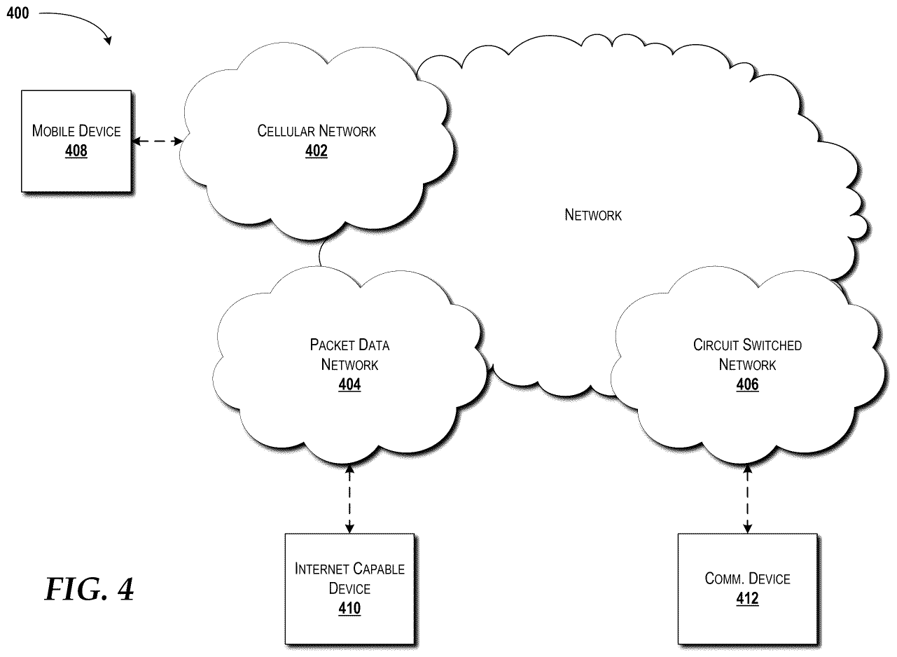

Turning now to FIG. 4, additional details of a network 400 are illustrated, according to an illustrative embodiment. The network 400 can include the 5G core network 102 and/or the other network(s) 140. The illustrated network 400 includes a cellular network 402, a packet data network 404, for example, the Internet, and a circuit switched network 406, for example, a publicly switched telephone network (“PSTN”). The cellular network 402 includes various components such as, but not limited to, base transceiver stations (“BTSs”), NodeB's or eNodeB's (“eNBs”), gNodeBs (“gNBs”), or the like; base station controllers (“BSCs”) radio network controllers (“RNCs”), or the like; an evolved packet core (“EPC”); mobile switching centers (“MSCs” or “MSSs”); session management functions (“SMFs); mobile management entities (“MMEs”); access and mobility management functions (“AMFs); authentication server functions (“AUSFs”), network slice selection functions (“NSSFs); network exposure functions (“NEFs”); policy control functions (“PCFs”); and various other functions in the user and control planes such as, for example, user plane functions (“UPFs), application functions (“AFs”), NF repository functions (“NRFs”), and the like; short message service centers (“SMSCs”); multimedia messaging service centers (“MMSCs”); home location registers (“HLRs”); home subscriber servers (“HSSs”); visitor location registers (“VLRs”); charging platforms; billing platforms; voicemail platforms; GPRS core network components; links to data networks (“DNs”) and/or other operator services, third party services, and/or the Internet; location service nodes, an IP Multimedia Subsystem (“IMS”); and the like. Of course, the cellular network 402 also can include various interfaces between various components, as is generally understood. The cellular network 402 also includes radios and nodes for receiving and transmitting voice, data, and combinations thereof to and from radio transceivers, networks, the packet data network 404, and the circuit switched network 406.

A mobile communications device 408, such as, for example, the user device 106, a cellular telephone, a user equipment, a mobile terminal, a PDA, a laptop computer, a handheld computer, and combinations thereof, can be operatively connected to the cellular network 402. The cellular network 402 can be configured as a 2G GSM network and can provide data communications via GPRS and/or EDGE. Additionally, or alternatively, the cellular network 402 can be configured as a 3G UMTS network and can provide data communications via the HSPA protocol family, for example, HSDPA, EUL (also referred to as HSUPA), and HSPA+. The cellular network 402 is also compatible with 4G mobile communications standards such as LTE, 5G mobile communications standards, 6G mobile communication standards, other mobile communications standards, and evolved and future mobile communications standards.

The packet data network 404 includes various devices, for example, servers, computers, databases, and other devices in communication with one another, as is generally known. The packet data network 404 devices are accessible via one or more network links. The servers often store various files that are provided to a requesting device such as, for example, a computer, a terminal, a smartphone, or the like. Typically, the requesting device includes software (a “browser”) for executing a web page in a format readable by the browser or other software. Other files and/or data may be accessible via “links” in the retrieved files, as is generally known. In some embodiments, the packet data network 404 includes or is in communication with the Internet. The circuit switched network 406 includes various hardware and software for providing circuit switched communications. The circuit switched network 406 may include, or may be, what is often referred to as a plain old telephone system (POTS). The functionality of a circuit switched network 406 or other circuit-switched network are generally known and will not be described herein in detail.

The illustrated cellular network 402 is shown in communication with the packet data network 404 and a circuit switched network 406, though it should be appreciated that this is not necessarily the case. One or more Internet-capable devices 410, for example, the user device 106, a PC, a laptop, a portable device, or another suitable device, can communicate with one or more cellular networks 402, and devices connected thereto, through the packet data network 404. It also should be appreciated that the Internet-capable device 410 can communicate with the packet data network 404 through the circuit switched network 406, the cellular network 402, and/or via other networks (not illustrated).

As illustrated, a communications device 412, for example, a telephone, facsimile machine, modem, computer, or the like, can be in communication with the circuit switched network 406, and therethrough to the packet data network 404 and/or the cellular network 402. It should be appreciated that the communications device 412 can be an Internet-capable device, and can be substantially similar to the Internet-capable device 410. In the specification, the network 400 is used to refer broadly to any combination of the networks 402, 404, 406. It should be appreciated that substantially all of the functionality described with reference to the RAN 104, the 5G core network 102, and/or the other network(s) 140 can be performed by the cellular network 402, the packet data network 404, and/or the circuit switched network 406, alone or in combination with other networks, network elements, and the like.

FIG. 5 is a block diagram illustrating a computer system 500 configured to provide the functionality described herein for providing the medical functions core manager 144 that generates medical network functions 146-164 that provide functionality, beyond that provided by the network functions 120-138 of the 5G core network 102, tailored for handling medical devices and/or medical-related traffic from user devices. The systems, devices, and other components disclosed herein, such as the server computer 142, components of the 5G core network 102, components of the other network(s) 140, or some combination thereof can be implemented, at least in part, using an architecture that is the same as or similar to the architecture of the computer system 500. The computer system 500 includes a processing unit 502, a memory 504, one or more user interface devices 506, one or more input/output (“I/O”) devices 508, and one or more network devices 510, each of which is operatively connected to a system bus 512. The system bus 512 can enable bi-directional communication between the processing unit 502, the memory 504, the user interface devices 506, the I/O devices 508, and the network devices 510.

The processing unit 502 may be a standard central processor that performs arithmetic and logical operations, a more specific purpose programmable logic controller (“PLC”), a programmable gate array, or other type of processor known to those skilled in the art and suitable for controlling the operation of the server computer. As used herein, the word “processor” and/or the phrase “processing unit” when used with regard to any architecture or system can include multiple processors or processing units distributed across and/or operating in parallel in a single machine or in multiple machines. Furthermore, processors and/or processing units can be used to support virtual processing environments. Processors and processing units also can include state machines, application-specific integrated circuits (“ASICs”), combinations thereof, or the like. Because processors and/or processing units are generally known, the processors and processing units disclosed herein will not be described in further detail herein.

The memory 504 communicates with the processing unit 502 via the system bus 512. In some embodiments, the memory 504 is operatively connected to a memory controller (not shown) that enables communication with the processing unit 502 via the system bus 512. The memory 504 includes an operating system 514 and one or more program modules 516. The operating system 514 can include, but is not limited to, members of the WINDOWS, WINDOWS CE, and/or WINDOWS MOBILE families of operating systems from MICROSOFT CORPORATION, the LINUX family of operating systems, the SYMBIAN family of operating systems from SYMBIAN LIMITED, the BREW family of operating systems from QUALCOMM CORPORATION, the MAC OS, iOS, and/or SONOMA families of operating systems from APPLE CORPORATION, the FREEBSD family of operating systems, the SOLARIS family of operating systems from ORACLE CORPORATION, other operating systems, and the like.

The program modules 516 may include various software and/or program modules described herein. In some embodiments, for example, the program modules 516 include the medical functions core manager 144, the network functions orchestrator 166, and the security model module 168. These and/or other programs can be embodied in computer-readable media containing instructions that, when executed by the processing unit 502, perform one or more of the methods 200 and 300 described in detail above with respect to FIGS. 2-3 and/or other functionality as illustrated and described herein. It can be appreciated that, at least by virtue of the instructions embodying the methods 200 and 300 and/or other functionality illustrated and described herein being stored in the memory 504 and/or accessed and/or executed by the processing unit 502, the computer system 500 is a special-purpose computing system that can facilitate providing the functionality illustrated and described herein. According to embodiments, the program modules 516 may be embodied in hardware, software, firmware, or any combination thereof. Although not shown in FIG. 5, it should be understood that the memory 504 also can be configured to store the replicated network functions 120′-138′ and the medical functions 170A-J and/or other data, if desired.

By way of example, and not limitation, computer-readable media may include any available computer storage media or communication media that can be accessed by the computer system 500. Communication media includes computer-readable instructions, data structures, program modules, or other data in a modulated data signal such as a carrier wave or other transport mechanism and includes any delivery media. The term “modulated data signal” means a signal that has one or more of its characteristics changed or set in a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Computer storage media includes only non-transitory embodiments of computer readable media as illustrated and described herein. Thus, computer storage media can include volatile and non-volatile, removable and non-removable media implemented in any method or technology for storage of information such as computer-readable instructions, data structures, program modules, or other data. Computer storage media includes, but is not limited to, RAM, ROM, Erasable Programmable ROM (“EPROM”), Electrically Erasable Programmable ROM (“EEPROM”), flash memory or other solid state memory technology, CD-ROM, digital versatile disks (“DVD”), or other optical storage, magnetic cassettes, magnetic tape, magnetic disk storage or other magnetic storage devices, or any other medium which can be used to store the desired information and which can be accessed by the computer system 500. In the claims, the phrase “computer storage medium” and variations thereof does not include waves or signals per se and/or communication media.

The user interface devices 506 may include one or more devices with which a user accesses the computer system 500. The user interface devices 506 may include, but are not limited to, computers, servers, personal digital assistants, cellular phones, or any suitable computing devices. The I/O devices 508 enable a user to interface with the program modules 516. In one embodiment, the I/O devices 508 are operatively connected to an I/O controller (not shown) that enables communication with the processing unit 502 via the system bus 512. The I/O devices 508 may include one or more input devices, such as, but not limited to, a keyboard, a mouse, or an electronic stylus. Further, the I/O devices 508 may include one or more output devices, such as, but not limited to, a display screen or a printer.

The network devices 510 enable the computer system 500 to communicate with other networks or remote systems via a network 518, such as the RAN 104, the 5G core network 102, and/or the other network(s) 140. Examples of the network devices 510 include, but are not limited to, a modem, a radio frequency (“RF”) or infrared (“IR”) transceiver, a telephonic interface, a bridge, a router, or a network card. The network 518 may include a wireless network such as, but not limited to, a Wireless Local Area Network (“WLAN”) such as a WI-FI network, a Wireless Wide Area Network (“WWAN”), a Wireless Personal Area Network (“WPAN”) such as BLUETOOTH, a Wireless Metropolitan Area Network (“WMAN”) such as a WiMAX network, or a cellular network. Alternatively, the network 518 may be a wired network such as, but not limited to, a Wide Area Network (“WAN”) such as the Internet, a Local Area Network (“LAN”) such as the Ethernet, a wired Personal Area Network (“PAN”), or a wired Metropolitan Area Network (“MAN”).

Turning now to FIG. 6, an illustrative mobile device 600 and components thereof will be described. In some embodiments, the user device 106 described above with reference to FIG. 1 can be configured as and/or can have an architecture similar or identical to the mobile device 600 described herein in FIG. 6. It should be understood, however, that the user device 106 may or may not include the functionality described herein with reference to FIG. 6. While connections are not shown between the various components illustrated in FIG. 6, it should be understood that some, none, or all of the components illustrated in FIG. 6 can be configured to interact with one another to carry out various device functions. In some embodiments, the components are arranged so as to communicate via one or more busses (not shown). Thus, it should be understood that FIG. 6 and the following description are intended to provide a general understanding of a suitable environment in which various aspects of embodiments can be implemented, and should not be construed as being limiting in any way.

As illustrated in FIG. 6, the mobile device 600 can include a display 602 for displaying data. According to various embodiments, the display 602 can be configured to display various graphical user interface (“GUI”) elements such as, for example, device names, login information, passwords, text, images, video, virtual keypads and/or keyboards, messaging data, notification messages, metadata, internet content, device status, time, date, calendar data, device preferences, map and location data, combinations thereof, and/or the like. The mobile device 600 also can include a processor 604 and a memory or other data storage device (“memory”) 606. The processor 604 can be configured to process data and/or can execute computer-executable instructions stored in the memory 606. The computer-executable instructions executed by the processor 604 can include, for example, an operating system 608, one or more applications 610 such as the medical functions client 107, other computer-executable instructions stored in a memory 606, or the like. In some embodiments, the applications 610 also can include a UI application (not illustrated in FIG. 6).

The UI application can interface with the operating system 608 to facilitate user interaction with functionality and/or data stored at the mobile device 600 and/or stored elsewhere. In some embodiments, the operating system 608 can include a member of the SYMBIAN OS family of operating systems from SYMBIAN LIMITED, a member of the WINDOWS MOBILE OS and/or WINDOWS PHONE OS families of operating systems from MICROSOFT CORPORATION, a member of the PALM WEBOS family of operating systems from HEWLETT PACKARD CORPORATION, a member of the BLACKBERRY OS family of operating systems from RESEARCH IN MOTION LIMITED, a member of the IOS family of operating systems from APPLE INC., a member of the ANDROID OS family of operating systems from GOOGLE INC., and/or other operating systems. These operating systems are merely illustrative of some contemplated operating systems that may be used in accordance with various embodiments of the concepts and technologies described herein and therefore should not be construed as being limiting in any way.

The UI application can be executed by the processor 604 to aid a user in entering content, creating device names, creating passwords, creating logins, selecting connections, requesting temporary connections, configuring settings, manipulating address book content and/or settings, multimode interaction, interacting with other applications 610, and otherwise facilitating user interaction with the operating system 608, the applications 610, and/or other types or instances of data 612 that can be stored at the mobile device 600. The data 612 can include applications or program modules. According to various embodiments, the data 612 can include, for example, presence applications, visual voice mail applications, messaging applications, text-to-speech and speech-to-text applications, add-ons, plug-ins, email applications, music applications, video applications, camera applications, location-based service applications, power conservation applications, game applications, productivity applications, entertainment applications, enterprise applications, combinations thereof, and the like. The applications 610, the data 612, and/or portions thereof can be stored in the memory 606 and/or in a firmware 614, and can be executed by the processor 604.

It can be appreciated that, at least by virtue of storage of the instructions corresponding to the applications 610 and/or other instructions embodying other functionality illustrated and described herein in the memory 606, and/or by virtue of the instructions corresponding to the applications 610 and/or other instructions embodying other functionality illustrated and described herein being accessed and/or executed by the processor 604, the mobile device 600 is a special-purpose mobile device that can facilitate providing the functionality illustrated and described herein. The firmware 614 also can store code for execution during device power up and power down operations. It can be appreciated that the firmware 614 can be stored in a volatile or non-volatile data storage device including, but not limited to, the memory 606 and/or a portion thereof.

The mobile device 600 also can include an input/output (“I/O”) interface 616. The I/O interface 616 can be configured to support the input/output of data, user information, organization information, presence status information, user IDs, passwords, and application initiation (start-up) requests. In some embodiments, the I/O interface 616 can include a hardwire connection such as a universal serial bus (“USB”) port, a mini-USB port, a micro-USB port, an audio jack, a PS2 port, an IEEE 1394 (“FIREWIRE”) port, a serial port, a parallel port, an Ethernet (RJ45 or RJ48) port, a telephone (RJ11 or the like) port, a proprietary port, combinations thereof, or the like. In some embodiments, the mobile device 600 can be configured to synchronize with another device to transfer content to and/or from the mobile device 600. In some embodiments, the mobile device 600 can be configured to receive updates to one or more of the applications 610 via the I/O interface 616, though this is not necessarily the case. In some embodiments, the I/O interface 616 accepts I/O devices such as keyboards, keypads, mice, interface tethers, printers, plotters, external storage, touch/multi-touch screens, touch pads, trackballs, joysticks, microphones, remote control devices, displays, projectors, medical equipment (e.g., stethoscopes, heart monitors, and other health metric monitors), modems, routers, external power sources, docking stations, combinations thereof, and the like. It should be appreciated that the I/O interface 616 may be used for communications between the mobile device 600 and a network device or local device.

The mobile device 600 also can include a communications component 618. The communications component 618 can be configured to interface with the processor 604 to facilitate wired and/or wireless communications with one or more networks such as the RAN 104, the 5G core network 102, and/or the other network(s) 140 described herein. In some embodiments, other networks include networks that utilize non-cellular wireless technologies such as WI-FI or WIMAX. In some embodiments, the communications component 618 includes a multimode communications subsystem for facilitating communications via the cellular network and one or more other networks.

The communications component 618, in some embodiments, includes one or more transceivers. The one or more transceivers, if included, can be configured to communicate over the same and/or different wireless technology standards with respect to one another. For example, in some embodiments one or more of the transceivers of the communications component 618 may be configured to communicate using GSM, CDMAONE, CDMA2000, LTE, and various other 2G, 2.5G, 3G, 4G, 5G, 6G, and greater generation technology standards. Moreover, the communications component 618 may facilitate communications over various channel access methods (which may or may not be used by the aforementioned standards) including, but not limited to, TDMA, FDMA, W-CDMA, OFDM, SDMA, and the like.

In addition, the communications component 618 may facilitate data communications using GPRS, EDGE, the HSPA protocol family including HSDPA, EUL or otherwise termed HSUPA, HSPA+, and various other current and future wireless data access standards. In the illustrated embodiment, the communications component 618 can include a first transceiver (“TxRx”) 620A that can operate in a first communications mode (e.g., GSM). The communications component 618 also can include an N^{th} transceiver (“TxRx”) 620N that can operate in a second communications mode relative to the first transceiver 620A (e.g., UMTS). While two transceivers 620A-N (hereinafter collectively and/or generically referred to as “transceivers 620”) are shown in FIG. 6, it should be appreciated that less than two, two, and/or more than two transceivers 620 can be included in the communications component 618.

The communications component 618 also can include an alternative transceiver (“Alt TxRx”) 622 for supporting other types and/or standards of communications. According to various contemplated embodiments, the alternative transceiver 622 can communicate using various communications technologies such as, for example, WI-FI, WIMAX, BLUETOOTH, infrared, infrared data association (“IRDA”), near field communications (“NFC”), other RF technologies, combinations thereof, and the like. In some embodiments, the communications component 618 also can facilitate reception from terrestrial radio networks, digital satellite radio networks, internet-based radio service networks, combinations thereof, and the like. The communications component 618 can process data from a network such as the Internet, an intranet, a broadband network, a WI-FI hotspot, an Internet service provider (“ISP”), a digital subscriber line (“DSL”) provider, a broadband provider, combinations thereof, or the like.

The mobile device 600 also can include one or more sensors 624. The sensors 624 can include temperature sensors, light sensors, air quality sensors, movement sensors, orientation sensors, noise sensors, proximity sensors, or the like. As such, it should be understood that the sensors 624 can include, but are not limited to, accelerometers, magnetometers, gyroscopes, infrared sensors, noise sensors, microphones, combinations thereof, or the like. Additionally, audio capabilities for the mobile device 600 may be provided by an audio I/O component 626. The audio I/O component 626 of the mobile device 600 can include one or more speakers for the output of audio signals, one or more microphones for the collection and/or input of audio signals, and/or other audio input and/or output devices.

The illustrated mobile device 600 also can include a subscriber identity module (“SIM”) system 628. The SIM system 628 can include a universal SIM (“USIM”), a universal integrated circuit card (“UICC”) and/or other identity devices. The SIM system 628 can include and/or can be connected to or inserted into an interface such as a slot interface 630. In some embodiments, the slot interface 630 can be configured to accept insertion of other identity cards or modules for accessing various types of networks. Additionally, or alternatively, the slot interface 630 can be configured to accept multiple subscriber identity cards. Because other devices and/or modules for identifying users and/or the mobile device 600 are contemplated, it should be understood that these embodiments are illustrative, and should not be construed as being limiting in any way.

The mobile device 600 also can include an image capture and processing system 632 (“image system”). The image system 632 can be configured to capture or otherwise obtain photos, videos, and/or other visual information. As such, the image system 632 can include cameras, lenses, charge-coupled devices (“CCDs”), combinations thereof, or the like. The mobile device 600 may also include a video system 634. The video system 634 can be configured to capture, process, record, modify, and/or store video content. Photos and videos obtained using the image system 632 and the video system 634, respectively, may be added as message content to an MMS message, email message, and sent to another mobile device. The video and/or photo content also can be shared with other devices via various types of data transfers via wired and/or wireless communication devices as described herein.

The mobile device 600 also can include one or more location components 636. The location components 636 can be configured to send and/or receive signals to determine a geographic location of the mobile device 600. According to various embodiments, the location components 636 can send and/or receive signals from global positioning system (“GPS”) devices, assisted-GPS (“A-GPS”) devices, WI-FI/WIMAX and/or cellular network triangulation data, combinations thereof, and the like. The location component 636 also can be configured to communicate with the communications component 618 to retrieve triangulation data for determining a location of the mobile device 600. In some embodiments, the location component 636 can interface with cellular network nodes, telephone lines, satellites, location transmitters and/or beacons, wireless network transmitters and receivers, combinations thereof, and the like. In some embodiments, the location component 636 can include and/or can communicate with one or more of the sensors 624 such as a compass, an accelerometer, and/or a gyroscope to determine the orientation of the mobile device 600. Using the location component 636, the mobile device 600 can generate and/or receive data to identify its geographic location, or to transmit data used by other devices to determine the location of the mobile device 600. The location component 636 may include multiple components for determining the location and/or orientation of the mobile device 600.

The illustrated mobile device 600 also can include a power source 638. The power source 638 can include one or more batteries, power supplies, power cells, and/or other power subsystems including alternating current (“AC”) and/or direct current (“DC”) power devices. The power source 638 also can interface with an external power system or charging equipment via a power I/O component 640. Because the mobile device 600 can include additional and/or alternative components, the above embodiment should be understood as being illustrative of one possible operating environment for various embodiments of the concepts and technologies described herein. The described embodiment of the mobile device 600 is illustrative, and should not be construed as being limiting in any way.

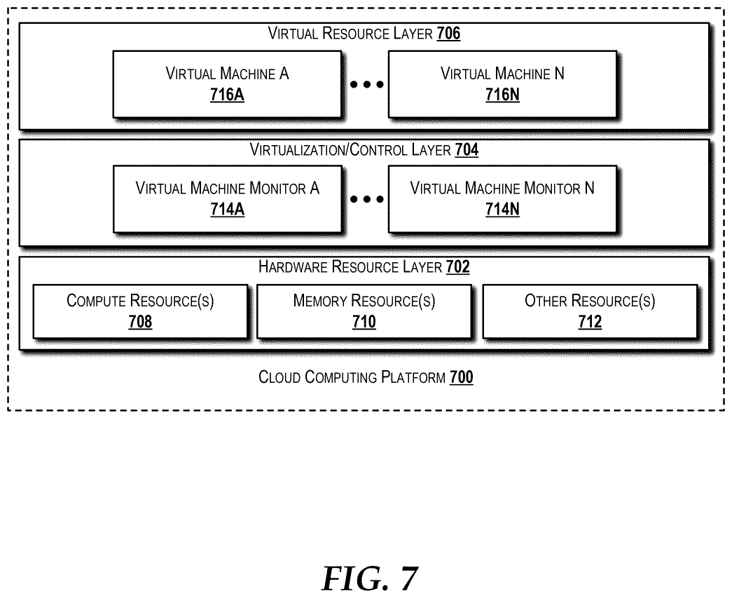

FIG. 7 illustrates an illustrative architecture for a cloud computing platform 700 that is capable of hosting some of the network functions of the 5G core network 102 and providing some of the functionality of the 5G core network 102, in accordance with various embodiments of the concepts and technologies disclosed herein. The cloud computing platform 700 thus may be utilized to execute any aspects of the software components presented herein. Those skilled in the art will appreciate that the illustrated cloud computing platform 700 is a simplification of only one possible implementation of an illustrative cloud computing platform, and as such, the illustrated cloud computing platform 700 should not be construed as being limiting in any way.

In the illustrated embodiment, the cloud computing platform 700 can include a hardware resource layer 702, a virtualization/control layer 704, and a virtual resource layer 706. These layers and/or other layers can be configured to cooperate with each other and/or other elements of a cloud computing platform 700 to perform operations as will be described in detail herein. While connections are shown between some of the components illustrated in FIG. 7, it should be understood that some, none, or all of the components illustrated in FIG. 7 can be configured to interact with one another to carry out various functions described herein. In some embodiments, the components are arranged so as to communicate via one or more networks such as, for example, the RAN 104, the 5G core network 102 and/or the other network(s) 140 illustrated and described hereinabove (not shown in FIG. 7). Thus, it should be understood that FIG. 7 and the following description are intended to provide a general understanding of a suitable environment in which various aspects of embodiments can be implemented, and should not be construed as being limiting in any way.

The hardware resource layer 702 can provide hardware resources. In the illustrated embodiment, the hardware resources can include one or more compute resources 707, one or more memory resources 710, and one or more other resources 712. The compute resource(s) 707 can include one or more hardware components that can perform computations to process data, and/or to execute computer-executable instructions of one or more application programs, operating systems, services, and/or other software including, but not limited to, the call transfer management service 122 illustrated and described herein.

According to various embodiments, the compute resources 707 can include one or more central processing units (“CPUs”). The CPUs can be configured with one or more processing cores. In some embodiments, the compute resources 707 can include one or more graphics processing units (“GPUs”). The GPUs can be configured to accelerate operations performed by one or more CPUs, and/or to perform computations to process data, and/or to execute computer-executable instructions of one or more application programs, operating systems, and/or other software that may or may not include instructions that are specifically graphics computations and/or related to graphics computations. In some embodiments, the compute resources 707 can include one or more discrete GPUs. In some other embodiments, the compute resources 707 can include one or more CPU and/or GPU components that can be configured in accordance with a co-processing CPU/GPU computing model. Thus, it can be appreciated that in some embodiments of the compute resources 707, a sequential part of an application can execute on a CPU and a computationally-intensive part of the application can be accelerated by the GPU. It should be understood that this example is illustrative, and therefore should not be construed as being limiting in any way.

In some embodiments, the compute resources 707 also can include one or more system on a chip (“SoC”) components. It should be understood that an SoC component can operate in association with one or more other components as illustrated and described herein, for example, one or more of the memory resources 710 and/or one or more of the other resources 712. In some embodiments in which an SoC component is included, the compute resources 707 can be or can include one or more embodiments of the SNAPDRAGON brand family of SoCs, available from QUALCOMM of San Diego, California; one or more embodiment of the TEGRA brand family of SoCs, available from NVIDIA of Santa Clara, California; one or more embodiment of the HUMMINGBIRD brand family of SoCs, available from SAMSUNG of Seoul, South Korea; one or more embodiment of the Open Multimedia Application Platform (“OMAP”) family of SoCs, available from TEXAS INSTRUMENTS of Dallas, Texas; one or more customized versions of any of the above SoCs; and/or one or more other brand and/or one or more proprietary SoCs.

The compute resources 707 can be or can include one or more hardware components arranged in accordance with an ARM architecture, available for license from ARM HOLDINGS of Cambridge, United Kingdom. Alternatively, the compute resources 707 can be or can include one or more hardware components arranged in accordance with an x86 architecture, such as an architecture available from INTEL CORPORATION of Mountain View, California, and others. Those skilled in the art will appreciate the implementation of the compute resources 707 can utilize various computation architectures and/or processing architectures. As such, the various example embodiments of the compute resources 707 as mentioned hereinabove should not be construed as being limiting in any way. Rather, implementations of embodiments of the concepts and technologies disclosed herein can be implemented using compute resources 707 having any of the particular computation architecture and/or combination of computation architectures mentioned herein as well as other architectures.

The memory resource(s) 710 can include one or more hardware components that can perform or provide storage operations, including temporary and/or permanent storage operations. In some embodiments, the memory resource(s) 710 can include volatile and/or non-volatile memory implemented in any method or technology for storage of information such as computer-readable instructions, data structures, program modules, or other data disclosed herein. Computer storage media is defined hereinabove and therefore should be understood as including, in various embodiments, random access memory (“RAM”), read-only memory (“ROM”), Erasable Programmable ROM (“EPROM”), Electrically Erasable Programmable ROM (“EEPROM”), flash memory or other solid state memory technology, CD-ROM, digital versatile disks (“DVD”), or other optical storage, magnetic cassettes, magnetic tape, magnetic disk storage or other magnetic storage devices, or any other medium that can be used to store data and that can be accessed by the compute resources 707, subject to the definition of “computer storage media” provided above (e.g., as excluding waves and signals per se and/or communication media as defined in this application). The other resource(s) 712 can include any other hardware resources that can be utilized by the compute resources(s) 707 and/or the memory resource(s) 710 to perform operations. The other resource(s) 712 can include one or more input and/or output processors (e.g., a network interface controller and/or a wireless radio), one or more modems, one or more codec chipsets, one or more pipeline processors, one or more fast Fourier transform (“FFT”) processors, one or more digital signal processors (“DSPs”), one or more speech synthesizers, combinations thereof, or the like.

The hardware resources operating within the hardware resource layer 702 can be virtualized by one or more virtual machine monitors (“VMMs”) 714A-714N (also known as “hypervisors;” hereinafter “VMMs 714”). The VMMs 714 can operate within the virtualization/control layer 704 to manage one or more virtual resources that can reside in the virtual resource layer 706. The VMMs 714 can be or can include software, firmware, and/or hardware that alone or in combination with other software, firmware, and/or hardware, can manage one or more virtual resources operating within the virtual resource layer 706.

The virtual resources operating within the virtual resource layer 706 can include abstractions of at least a portion of the compute resources 707, the memory resources 710, the other resources 712, or any combination thereof. These abstractions are referred to herein as virtual machines (“VMs”). In the illustrated embodiment, the virtual resource layer 706 includes VMs 716A-716N (hereinafter “VMs 716”).

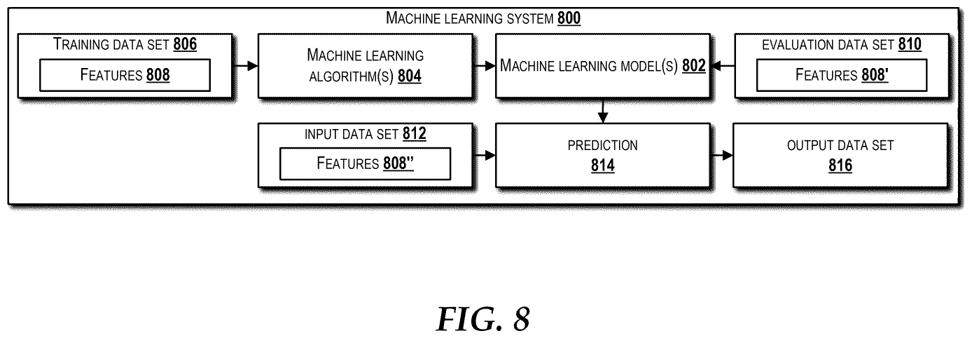

Turning now to FIG. 8, a machine learning system 800 capable of implementing aspects of the embodiments disclosed herein will be described. The machine learning system 800 can be used to train the security model module 168. Accordingly, the server computer 142 can include the machine learning system 800 or can be in communication with the machine learning system 800.

The illustrated machine learning system 800 includes one or more machine learning models 802. The machine learning models 802 can include unsupervised, supervised, and/or semi-supervised learning models. The machine learning model(s) 802 can be created by the machine learning system 800 based upon one or more machine learning algorithms 804. The machine learning algorithm(s) 804 can be any existing, well-known algorithm, any proprietary algorithms, or any future machine learning algorithm. Some example machine learning algorithms 804 include, but are not limited to, time series autoregression, Seasonal and Trend Decomposition, Seasonal and Trend Decomposition using Loess (locally estimated scatterplot smoothing), Bayesian Estimator of Abrupt Change, Seasonality, Trend (BEAST), neural networks, gradient descent, linear regression, logistic regression, linear discriminant analysis, decision trees, Naive Bayes, K-nearest neighbor, learning vector quantization, support vector machines, principal component analysis, and the like. Those skilled in the art will appreciate the applicability of various machine learning algorithms 804 based upon the problem(s) to be solved by machine learning via the machine learning system 800.

The machine learning system 800 can control the creation of the machine learning models 802 via one or more training parameters (also referred to as “tuning parameters”). In some embodiments, the training parameters are selected variables or factors at the direction of an enterprise, for example. Alternatively, in some embodiments, the training parameters are automatically selected based upon data provided in one or more training data sets 806. The training parameters can include, for example, a learning rate where relevant such as when a classification algorithm is utilized, a model size, a number of training passes, data shuffling, regularization, and/or other training parameters known to those skilled in the art.

The learning rate is a training parameter defined by a constant value. The learning rate affects the speed at which the machine learning algorithm 804 converges to the optimal weights. The machine learning algorithm 804 can update the weights for every data example included in the training data sets 806. The size of an update is controlled by the learning rate. A learning rate that is too high might prevent the machine learning algorithm 804 from converging to the optimal weights. A learning rate that is too low might result in the machine learning algorithm 804 requiring multiple training passes to converge to the optimal weights.

The model size is regulated by the number of input features (“features”) 808 in the training data sets 806. The training data sets 806 and evaluation data sets 810 discussed further below may be selected based on an appropriate training/test split for training and evaluation, such as an 80/20 split.

The number of training passes indicates the number of training passes that the machine learning algorithm 804 makes over the training data sets 806 during the training process. The number of training passes can be adjusted based, for example, on the size of the training data sets 806, with larger training data sets being exposed to fewer training passes in consideration of time and/or resource utilization. The performance of the resultant machine learning model 802 can be increased by multiple training passes.

Data shuffling is a training parameter designed to prevent the machine learning algorithm 804 from reaching false optimal weights due to the order in which data contained in the training data sets 806 is processed. For example, data provided in rows and columns might be analyzed first row, second row, third row, etc., and thus an optimal weight might be obtained well before a full range of data has been considered. By data shuffling, the data contained in the training data sets 806 can be analyzed more thoroughly and mitigate bias in the resultant machine learning model 802.

Regularization is a training parameter that helps to prevent the machine learning model 802 from memorizing training data from the training data sets 806. In other words, the machine learning model 802 fits the training data sets 806, but the predictive performance of the machine learning model 802 is not acceptable. Regularization helps the machine learning system 800 avoid this overfitting/memorization problem by adjusting extreme weight values of the features 808. For example, a feature that has a small weight value relative to the weight values of the other features in the training data sets 806 can be adjusted to zero.

The machine learning system 800 can determine model accuracy, recall, precision, receiver operating characteristic (“ROC”) area under the curve (“AUC”), and/or other desired metrics after training by using the training data sets 806 with some of the features 808 and testing the machine learning model 802 with unseen evaluation data sets 810 containing the same features 808′ in the training data sets 806. This also prevents the machine learning model 802 from simply memorizing the data contained in the training data sets 806, which can overfit the data. The optimal or desired machine learning system 800 is reached when a target model accuracy or other desired metric threshold is met, which is understood through a model evaluation process in examining model performance on the evaluation data set 810. Once a machine learning model 802 has reached the desired metric threshold or optimal performance, the machine learning model 802 is considered ready for deployment.

After deployment, the machine learning model 802 can perform a prediction operation (“prediction”) 814 with an input data set 812 having the same features 808″ as the features 808 in the training data sets 806 and the features 808′ of the evaluation data sets 810. The results of the prediction 814 are included in an output data set 816 consisting of predicted data. The machine learning model 802 can perform other operations, such as regression, classification, and others. As such, the example illustrated in FIG. 8 should not be construed as being limiting in any way.

Based on the foregoing, it should be appreciated that systems and methods for enabling management of calls and call transfers between linked devices to provide optimal connectivity for a call have been disclosed herein. Although the subject matter presented herein has been described in language specific to computer structural features, methodological and transformative acts, specific computing machinery, and computer-readable media, it is to be understood that the concepts and technologies disclosed herein are not necessarily limited to the specific features, acts, or media described herein. Rather, the specific features, acts and mediums are disclosed as example forms of implementing the concepts and technologies disclosed herein.

The subject matter described above is provided by way of illustration only and should not be construed as limiting. Various modifications and changes may be made to the subject matter described herein without following the example embodiments and applications illustrated and described, and without departing from the true spirit and scope of the embodiments of the concepts and technologies disclosed herein.

1. A system comprising:
a processor; and
a memory that stores computer-executable instructions for a medical functions core manager that, when executed by the processor, cause the processor to perform operations comprising
determining functionalities of a plurality of network functions of a next generation core network,
duplicating the functionalities of the plurality of network functions to generate replicated network functions,
integrating each of the replicated network functions with a corresponding medical function to generate a plurality of medical network functions, and
deploying the plurality of medical network functions to the next generation core network.

2. The system of claim 1, wherein the plurality of medical network functions provide functionalities that extend beyond the functionalities of the plurality of network functions.

3. The system of claim 1, wherein data from a user device determined to be a medical device is routed through at least a portion of the plurality of medical network functions instead of the plurality of network functions of the next generation core network.

4. The system of claim 3, wherein the user device is determined to be a medical device based, at least in part, on the data comprising a medical flag.

5. The system of claim 1, wherein data determined to be associated with medical information is routed through at least a portion of the plurality of medical network functions instead of the plurality of network functions of the next generation core network.

6. The system of claim 1, wherein the operations further comprise launching a plurality of virtual machines onto the next generation core network, and wherein one of the plurality of virtual machines is launched in association with each of the plurality of network functions.

7. The system of claim 6, wherein deploying the plurality of medical network functions to the next generation core network comprises deploying one of the plurality of medical network functions to each of the plurality of virtual machines launched in association with a corresponding one of the plurality of network functions.

8. A method comprising:
determining, by a system comprising a processor executing a medical functions core manager, functionalities of a plurality of network functions of a next generation core network;
duplicating, by the system, the functionalities of the plurality of network functions to generate replicated network functions;
integrating, by the system, each of the replicated network functions with a corresponding medical function to generate a plurality of medical network functions; and
deploying, by the system, the plurality of medical network functions to the next generation core network.

9. The method of claim 8, wherein the plurality of medical network functions provide functionalities that extend beyond the functionalities of the plurality of network functions.

10. The method of claim 8, wherein data from a user device determined to be a medical device is routed through at least a portion of the plurality medical network functions instead of the plurality of network functions of the next generation core network.

11. The method of claim 10, wherein the user device is determined to be a medical device based, at least in part, on the data comprising a medical flag.

12. The method of claim 8, wherein data determined to be associated with medical information is routed through the plurality of medical network functions instead of the plurality of network functions of the next generation core network.

13. The method of claim 8, further comprising launching a plurality of virtual machines onto the next generation core network, wherein one of the plurality of virtual machines is launched in association with each of the plurality of network functions.

14. The method of claim 13, wherein deploying the plurality of medical network functions to the next generation core network comprises deploying one of the plurality of medical network functions to each of the plurality of virtual machines launched in association with a corresponding one of the plurality of network functions.

15. A computer storage medium having computer-executable instructions for a medical functions core manager stored thereon that, when executed by a processor of a system, cause the processor to perform operations comprising:
determining functionalities of a plurality of network functions of a next generation core network;
duplicating the functionalities of the plurality of network functions to generate replicated network functions;
integrating each of the replicated network functions with a corresponding medical function to generate a plurality of medical network functions; and
deploying the plurality of medical network functions to the next generation core network.

16. The computer storage medium of claim 15, wherein the plurality of medical network functions provide functionality that extends beyond the functionalities of the plurality of network functions.

17. The computer storage medium of claim 15, wherein data from a user device determined to be a medical device is routed through at least a portion of the plurality of medical network functions instead of the plurality of network functions of the next generation core network.

18. The computer storage medium of claim 17, wherein the user device is determined to be a medical device based, at least in part, on the data comprising a medical flag.

19. The computer storage medium of claim 15, wherein data determined to be associated with medical information is routed through at least a portion of the plurality of medical network functions instead of the plurality of network functions of the next generation core network.

20. The computer storage medium of claim 15, wherein the operations further comprise launching a plurality of virtual machines onto the next generation core network, wherein one of the plurality of virtual machines is launched in association with each of the plurality of network functions, and wherein deploying the plurality of medical network functions to the next generation core network comprises deploying one of the plurality of medical network functions to each of the plurality of virtual machines launched in association with a corresponding one of the plurality of network functions.
